(19) United States
(12) Patent Application Publication
(10) Pub. No.: US 2008/0132533 A1
(43) Pub. Date: Jun. 5, 2008

(54) Solid Dispersion Comprising Tacrolimus and Entericcoated Macromolecule

(75) Inventors: Dae II Yeom, Gyeonggi-do (KR); Hun Sik Wang, Gyeonggi-do (KR); Jun Sang Park, Gyeonggi-do (KR); Woo Heon Song, Gyeonggi-do (KR)

Correspondence Address: SUGHRUE MION, PLLC, 2100 PENNSYLVANIA AVENUE, N.W., SUITE 800, WASHINGTON, DC, 20037, US

(73) Assignee: GL PharmTech Corp., Seongnama-si (KR)

(21) Appl. No.: 11/814,583

(22) PCT Filed: Dec. 30, 2005
(86) PCT No.: PCT/KR05/04669

§ 371 Date: Jul. 24, 2007

(30) Foreign Application Priority Data
Jan. 25, 2005 (KR) 10-2005-0006872

(51) Int. Cl. A61K 31/4353; A61P 31/04

(52) U.S. Cl. 514/291

(57) ABSTRACT

This invention relates to a solid dispersion formulation comprising tacrolimus and enteric polymer. The solid dispersion formulation may contribute to better stability of tacrolimus preparation under high temperature and humidity condition through reduced recrystallization rate of tacrolimus. In addition, the solid dispersion formulation releases tacrolimus immediately in aqueous media and elevated solubility level maintains for certain period of time and that way the formulation may also enhance the bioavailability and oral absorption rate of tacrolimus.

### TECHNICAL FIELD

This invention relates to a solid dispersion formulation comprising tacrolimus and enteric polymer. This invention is designed to incorporate tacrolimus, a poorly water-soluble drug, into a solid dispersion formulation containing enteric polymer, thus enhancing the stability of tacrolimus formulation by reducing the recrystallization rate of amorphous tacrolimus under high temperature and high humidity condition. The formulation releases tacrolimus immediately in aqueous media and also maintains elevated solubility level for a certain period of time to ensure the maximized bioavailability and oral absorption rate of tacrolimus.

### BACKGROUND ART

Tacrolimus, isolated from Streptomyces tsukubaensis, is a macrolide antibiotic with immunosuppressive and antimicrobial activities. Currently, tacrolimus has been clinically in use as prophylaxis against organ rejection after liver, bone marrow and renal transplantation. But due to the poor solubility of tacrolimus in water (1˜2 [symbol] /mL), its bioavailability is extremely low when administered orally.

In an attempt to enhance the oral absorption rate and bioavailability of tacrolimus, a variety of solid dispersion formulations with rapid solubility profile have been developed.

The Korean Patent Examined Publication No. 1995-7209 discloses a solid dispersion formulation of tacrolimus in an amorphous form using a water-soluble polymer.

Similarly, the Korean Patent Unexamined Publication No. 2000-57242 discloses a solid dispersion formulation of tacrolimus in an amorphous form using surfactants in an effort to improve a carrier function and dissolution rate of tacrolimus.

However, it is reported that as a drug formulated into an amorphous form is converted to crystalline form with the passage of time, its solubility decreases again (Solid dispersion of poorly water-soluble drugs: Early promises, subsequent problems, and recent breakthroughs, J. Pharm. Sci., 1999, 88(10), p. 1058).

The solid dispersion formulation of tacrolimus, which was described in the Korean Patent Examined Publication No. 1995-7209, is a rapidly soluble preparation with better solubility profile. However, a water-soluble polymer in the solid dispersion allows for water to infiltrate into the formulation easily due to the intrinsic hygroscopic property of the polymer, thus making amorphous form unstable in the presence of moisture.

Since tacrolimus is a compound with relatively high molecular weight of over 800 and with a complicated chemical structure, its amorphous form recrystallizes easily with the passage of time, when compared to a compound with lower molecular weight, thus leading to further reduction of solubility.

In particular, the recrystallization of the disclosed tacrolimus solid dispersion formulation affects the physical stability of tacrolimus preparation severely and occurs easily during the summer season of relatively high temperature and humidity.

Therefore, in the Korean Patent Examined Publication No. 1995-7209, a multiple anti-humidity blister package has to be adopted to block the infiltration of water from outer environment and to overcome the recrystallization of active ingredient under higher temperature and humidity condition.

Prograf® capsules, a commercial capsule formulation, are available in a multiple anti-humidity blister package with an aluminum bag, but their stability issue remains to be problematic, when being left for a long-term period after the first unsealing.

If the conventional solid dispersion formulation is added to the aqueous medium, a transient solubility-elevating effect is only noticeable and with the passage of time, there is a significant decrease in solubility. Since it takes at least 2-6 hours for dosage forms to pass through the whole small intestine following oral administration, the transient elevation of solubility only cannot ensure the maximization of the oral absorption of drugs in the body. Furthermore, in the case of a drug absorbed in all part of small intestine, its elevated solubility needs to be maintained for at least 4 hours by taking account of their retention time in the small intestine.

Therefore, the solid dispersion formulation of tacrolimus should inhibit the recrystallization and maintain the elevated solubility for a longer period of time in the body, instead of formulating a solid dispersion only for enhancing solubility.

Meanwhile, an enteric polymer is not soluble in acidic environment of stomach but soluble in alkaline condition of intestine, when applied to human body. Such polymer is usually dissolved at above pH 5.05 and is poorly soluble in water, even if some differences exist.

The conventional preparation containing nifedipine and an enteric polymer is reported to increase the solubility of nifedipine (Drug Development and Industrial Pharmacy 30(1), 9-17, 2004).

The Korean Patent No. 179343 also discloses a rapidly soluble and pH independent preparation through the mixture of a pH-dependent, poorly water-soluble drug with an enteric polymer.

As described above, a series of technologies designed to increase the solubility of poorly water-soluble drugs were already disclosed and it is thought that the nature and effect of a drug product is greatly affected by the interaction between a drug and a matrix or a carrier, which disperse a drug into molecular state. However, since the compatibility between a drug and a carrier is not easily predictable, each formulation should be regarded as different thing depending on the nature of each drug (Phase behavior of amorphous molecular dispersions 1: Determination of the degree and mechanism of solid solubility, Pharm. Res., 2004, 21(9), p. 1598).

In this respect, it is not predicted that all mixtures of an enteric polymer with poorly water-soluble drugs can necessarily elevate their solubility.

The inventors make endeavor to intensively improve the stability of tacrolimus preparation by slowing the recrystallization rate of tacrolimus and to maintain the elevated solubility for certain period of time. As a result, the inventors discovered that the solid dispersion formulation containing tacrolimus and enteric polymer may meet the above objects and thus, consummated this invention.

### DISCLOSURE OF INVENTION

### Technical Problem

An object of this invention is to provide a solid dispersion formulation containing tacrolimus with the excellent combination of properties, including better stability of tacrolimus preparation by reducing the recrystallization rate of amorphous tacrolimus and improved bioavailability and oral absorption rate of tacrolimus preparation thanks to solubility enhancement of tacrolimus and its longer retention time up to 4 hours.

### Technical Solution

To achieve the aforementioned objective, this invention provides a solid dispersion formulation containing tacrolimus and enteric polymer.

The solid dispersion formulation of this invention is described as set forth hereunder.

The solid dispersion formulation of this invention contains tacrolimus and enteric polymer as active ingredients.

Tacrolimus may be isolated from a culture solution of Streptomyces sp. strain via fermentation, or it may be purchased from Fujisawa Co. of Japan.

The enteric polymer, when administered orally, is not soluble in acidic condition of stomach, but soluble in higher pH condition in small intestine. Although absorption behavior is more or less different depending upon the kinds of enteric materials, the enteric polymer is commonly dissolved in more than pH 5.0 and is poorly soluble in water.

The enteric polymer of this invention is not limited and the examples include Shellac, cellulose acetate phthalate, hydroxypropylmethylcellulose phthalate, acrylate copolymer (methacrylic acid/methyl methacrylate copolymer or methacrylic acid/ethyl methacrylate copolymer, brandname: Eudragit L), polyvinyl acetate phthalate, hydroxypropylmethylcellulose acetate succinate, carboxymethylethylcellulose, and other enteric polymers. It is preferred to employ hydroxypropylmethylcellulose phthalate, acrylate copolymer, hydroxypropylmethylcellulose acetate succinate, and carboxymethylethylcellulose, and among them, hydroxypropylmethylcellulose phthalate is the most preferred enteric polymer.

The amount of enteric polymer is not restrictive and is any one, by which tacrolimus can be dispersed. The preferable weight ratio of tacrolimus and enteric polymer is 1:0.5˜1.2, and more preferable one is 1:0.8˜1.2.

The solid dispersion formulation can be prepared by a conventional method, for example;

1) a step to dissolve tacrolimus and enteric polymer in an organic solvent;

2) a step to suspend an excipient in the solution selectively;

3) a step to evenly disperse the resulting solution in the additives such as diluent, disintegrant, and the like, if necessary, and

4) a step to remove the solvent from the dispersed solution by a conventional method.

The following variants in the above 3) dispersion step should be coincided with the object of this invention: viscosity and solid content of solution, flowability, particle size, distribution of particle size, inlet air temperature, outlet air temperature, and spraying pressure.

Any organic solvents, which are capable of dissolving tacrolimus, may be employed and the examples include alcohols such as methanol, ethanol, propanol and isopropyl alcohol, ethyl acetate and acetone. These organic solvents may be used in a single or mixed form, and if necessary, water may be mixed with them.

The selectively used excipients and disintegrants may be commonly available in the field of pharmaceutical manufacture. They can be used in a single or mixed form. The examples of such excipient and diluent include lactose, sucrose, starch, mannitol, and inorganic salt. The examples of such disintegrant include croscarmellose sodium, calcium carboxymethylcellulose, low-substituted hydroxypropylcellulose, and sodium starch glycolate.

The solid dispersion formulation of this invention can be used by itself as an oral medication and also may be available for various other dosage forms such as powders, fine granules, granules, tablets, and capsules and the like. If necessary, pharmaceutically acceptable additives may be mixed with the solid dispersion formulations.

The composition of this invention is prepared in such a manner that the therapeutic dose of tacrolimus is contained. The therapeutic dose of tacrolimus may vary depending upon individual patients, but the daily dose of tacrolimus should be administered to an adult patient (60 kg) in the range of 0.1˜500 mg, preferably in the range of 0.5˜100 mg.

Generally, the amount of tacrolimus in the composition of this invention is contained by the weight ratio of 0.1˜75%, although the contents of therapeutically effective tacrolimus may vary depending upon individual patients or dosage forms.

The solid dispersion formulation of this invention containing enteric polymer serves to improve the stability of tacrolimus preparation under relatively high temperature and humidity condition by reducing the recrystallization rate of amorphous tacrolimus. Furthermore, the solid dispersion formulation allows tacrolimus to be released rapidly and maintained the elevated solubility for at least 4 hours, thus it can make tacrolimus be absorbed into all part of small intestine and maximize the bioavailability and oral absorption rate of tacrolimus.

### Advantageous Effects

The solid dispersion formulation of this invention containing an enteric polymer plays an important role to enhance the stability of tacrolimus preparation through the reduced recrystallization rate of amorphous tacrolimus, even under high temperature and humidity condition.

Further, the solid dispersion formulation of this invention allows for the initial solubility of tacrolimus to be increased rapidly, and stabilizes its elevated solubility for at least 4 hours. Therefore, an object of this invention is to provide the solid dispersion formulation of tacrolimus that may ensure better stability of tacrolimus preparation and maximize its bioavailability and oral absorption rate.

### BRIEF DESCRIPTION OF THE DRAWINGS

FIG. 1 is a graph showing the changes of solubility in water at 37° C. relating to Comparative Example 1, tacrolimus powder, and Examples 1˜2.

FIG. 2 is a graph showing the changes of solubility in water at 37° C. relating to Examples 4˜6.

### BEST MODE FOR CARRYING OUT THE INVENTION

This invention will now be described by reference to the following examples and experimental examples which are merely illustrative and which are not to be construed as a limitation of the scope of this invention.

### EXAMPLE 1

### Preparation of a Solid Dispersion Formulation Containing Tacrolimus and Hydroxypropylmethylcellulose Phthalate in the Weight Ratio of 1:1

1.0 g of tacrolimus was dissolved in 95% acetone (35 mL) and was added to 1.0 g of hydroxypropylmethylcellulose phthalate (brandname: HPMCP HP-50, Shin-Etsu Chemical). Then the solid content was completely dissolved. 2.8 g of calcium carboxymethylcellulose was suspended in the solution. This suspension was added to 133.8 g of lactose and the mixture was kneaded. Then, the solvent was evaporated under reduced pressure for 14 hours using a vacuum drier. The dried material was screened through a 30-mesh sieve to obtain a solid dispersion formulation.

### EXAMPLE 2

### Preparation of a Solid Dispersion Formulation Containing Tacrolimus and Hydroxypropylmethylcellulose Phthalate in the Weight Ratio of 1:0.8

A solid dispersion formulation was obtained in the same manner as described in Example 1, except that 0.8 g of hydroxypropylmethylcellulose phthalate (brandname: HPMCP HP-50, Shin-Etsu Chemical) was used instead of 1.0 g.

### EXAMPLE 3

### Preparation of a Solid Dispersion Formulation Containing Tacrolimus and Hydroxypropylmethylcellulose Acetate Succinate

A solid dispersion formulation was obtained in the same manner as described in Example 1, except that 1.0 g of hydroxypropylmethylcellulose acetate succinate (brandname: AQOAT AS-LF, Shin-Etsu Chemical) was used instead of hydroxypropylmethylcellulose phthalate.

### EXAMPLE 4

### Preparation of a Solid Dispersion Formulation Containing Tacrolimus and Carboxymethylethylcellulose

1.0 g of tacrolimus was dissolved in 93% ethanol (35 mL) and was added to 1.0 g of carboxymethylethylcellulose (brandname: CMEC, Freund Ind.). Then the solid content was completely dissolved. 2.8 g of calcium carboxymethylcellulose was suspended in the solution. This suspension was added to 133.8 g of lactose and the mixture was kneaded. Then, the solvent was evaporated under reduced pressure for 14 hours using a vacuum drier. The dried material was screened through a 30-mesh sieve to obtain a solid dispersion formulation.

### EXAMPLE 5

### Preparation of a Solid Dispersion Formulation Containing Tacrolimus and Acrylate

A solid dispersion formulation was obtained in the same manner as described in Example 1, except that 1.0 g of acrylate polymer (brandname: Eudragit L100-55, Degussa) was used instead of hydroxypropylmethylcellulose phthalate.

### EXAMPLE 6

### Preparation of a Solid Dispersion Formulation Containing Tacrolimus and Hydroxypropylmethylcellulose Phthalate in the Weight Ratio of 1:1.5

1 g of tacrolimus was dissolved in 95% acetone (35 mL) and was added to 1.5 g of hydroxypropylmethylcellulose phthalate (brandname: HPMCP HP-50, Shin-Etsu Chemical), and then the solid content was completely dissolved. Then, the solution was dried under reduced pressure for 15 hours using a vacuum drier. The dried material was screened through a 30-mesh sieve to obtain a solid dispersion formulation.

### COMPARATIVE EXAMPLE

### Prograf® Capsule

Prograf® capsules, a commercial capsule formulation (Fujisawa Co.), were used as a reference formulation.

### EXPERIMENTAL EXAMPLE 1

### Stability Test Under High Temperature and Humidity Condition

The stability test for solid dispersion formulations, prepared from Examples 1˜5, and Prograf® capsules, was performed to compare the physical stability of tacrolimus formulations.

1. Testing Procedure

The solid dispersion formulations were exposed to the outer environment under 40° C. and 75% RH storage condition. Then, specimens were collected at day 0 (initial), 12, and 20 for solubility testing.

The solubility test was performed in 100 mL of water at 37° C. using Vankel 100 ml vessel and paddle system at 200 rpm. The excessive specimens were loaded, and the samples for analysis were collected after 0.5, 1, and 2 hours and the solubility of tacrolimus was analyzed by high performance liquid chromatography.

A dissolution test for the specimens from Examples 1˜2 was performed in accordance with method II of the Korean Pharmacopoeia (900 mL of water at 37° C., 50 rpm). An appropriate amount of samples, corresponding to 1 mg of drug, were applied.

The specimens were collected after 60 minutes and analyzed.

2. Results

As shown in Tables 1˜2, the solubility of Comparative example 1 decreased by about 20˜50% under the test condition (40° C. and 75% relative humidity), On the other hand, the specimens from Examples 1˜5 showed relatively small solubility change (less than 10%) under the same test condition.

Furthermore, The results in Table 3 show that the dissolution rates of specimens from Examples 1˜2 were reduced by less than 8%, while that of Comparative example 1 decreased by more than 15%.

In this context, the results of stability test demonstrate that the solid dispersion formulation of this invention contributed much to slowing the recrystallization rate of amorphous tacrolimus under high temperature and humidity condition, thus enhancing stability of tacrolimus preparation.

TABLE 1
Changes in solubility of Comparative example 1 and Example 1~2 under
40° C./75% RH open condition

| | | 30 min | | 60 min | | 120 min | |
|---|---|---|---|---|---|---|---|
| | | Solubility | | Solubility | | Solubility | |
| | Day | (μg/mL) | Change (%) | (μg/mL) | Change (%) | (μg/mL) | Change (%) |
| Comparative | 0 | 38.52 | | 47.57 | | 50.00 | |
| Example 1 | 12 | 27.13 | −29.57 | 37.07 | −22.07 | 41.63 | −16.74 |
| | 20 | 22.73 | −40.99 | 24.87 | −47.72 | 40.15 | −19.70 |
| Example 1 | 0 | 50.26 | | 50.48 | | 51.61 | |
| | 12 | 48.72 | −3.06 | 55.27 | +9.47 | 49.66 | −3.77 |
| | 20 | 55.04 | +0.79 | 54.37 | +0.94 | 53.95 | +0.91 |
| Example 2 | 0 | 54.23 | | 55.16 | | 55.92 | |
| | 12 | 51.67 | −4.72 | 59.17 | +7.28 | 54.31 | −2.88 |
| | 20 | 59.14 | +9.05 | 59.87 | +8.54 | 56.09 | +0.29 |

TABLE 2
Changes in solubility of Example 3~5 under 40° C./75% RH open condition

| | | 30 min | | 60 min | | 120 min | |
|---|---|---|---|---|---|---|---|
| | | Solubility | | Solubility | | Solubility | |
| | Day | (μg/mL) | Change (%) | (μg/mL) | Change (%) | (μg/mL) | Change (%) |
| Example 3 | 0 | 43.76 | | 43.24 | | 43.44 | |
| | 12 | 43.39 | −0.85 | 44.50 | +2.91 | 45.06 | +3.75 |
| Example 4 | 0 | 46.36 | | 47.91 | | 47.42 | |
| | 12 | 49.37 | +6.50 | 48.21 | +0.63 | 46.80 | −1.30 |
| Example 5 | 0 | 25.87 | | 29.36 | | 29.66 | |
| | 12 | 29.65 | +14.61 | 31.17 | +6.18 | 31.53 | +6.33 |

TABLE 3
Changes in dissolution rate of Comparative example 1 and
Example 1~2 under 40° C./75% RH open condition (mean ± SD)

| | Day 0 | Day 12 (change %) | Day 20 (change %) |
|---|---|---|---|
| Comparative | 86.36 ± 5.49 | 71.32 ± 4.60 | 67.90 ± 1.69 |
| Example 1 | | (−17.42) | (−21.38) |
| Example 1 | 85.96 ± 2.61 | 83.20 ± 0.38 | 83.12 ± 2.23 |
| | | (−3.21) | (−3.31) |
| Example 2 | 87.45 ± 0.34 | 81.04 ± 1.61 | 81.95 ± 1.77 |
| | | (−7.33) | (−6.29) |

### EXPERIMENTAL EXAMPLE 2

### Dynamic Solubility Test

The dynamic solubility test for tacrolimus powder (control), Prograf (Comparative example 1), and solid dispersion formulations prepared from Examples 1˜6 (except Example 3), was performed to investigate the solubility changes of tacrolimus in water by the time intervals.

1. Testing Procedure

The solubility test was performed in 100 mL of water at 37° C. using Vankel 100 ml vessel and paddle system at 200 rpm. The excessive specimens were loaded and appropriate aliquots were collected after 0.5, 1, 2, 4, and 8 hours, and the samples were analyzed by high performance liquid chromatography.

2. Results

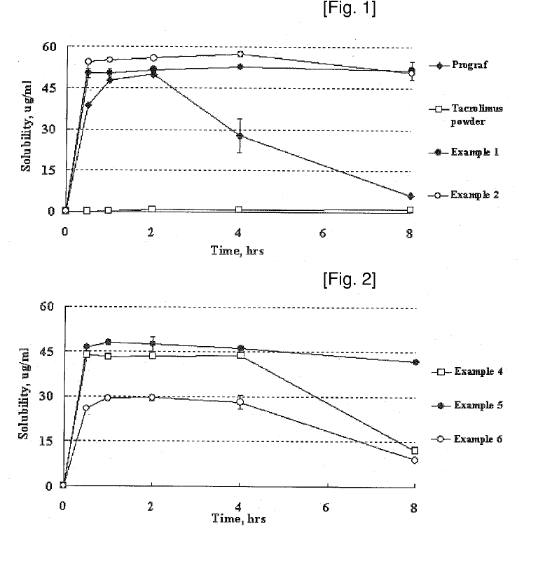

As shown in Table 4, the solubility enhancement of Examples of this invention was higher compared to that of Comparative example 1 at an initial stage. The solubility of tacrolimus of the Comparative example 1 was drastically reduced after 2 hours. On the other hand, that of Examples of this invention showed a constant solubility lasted up to at least 4 hours. (FIGS. 1, 2)

TABLE 4
Solubility of tacrolimus (μg/mL, mean ± SD)

| | Time (hr) | | | | |
|---|---|---|---|---|---|
| | 0.5 | 1 | 2 | 4 | 8 |
| Control | 0.00 ± 0.00 | 0.28 ± 0.05 | 0.68 ± 0.02 | 0.74 ± 0.01 | 0.96 ± 0.05 |
| Comparative | 38.52 ± 0.13 | 47.57 ± 0.94 | 50.00 ± 0.62 | 27.72 ± 6.19 | 6.23 ± 0.23 |
| Example 1 | | | | | |
| Example 1 | 50.26 ± 1.59 | 50.48 ± 1.37 | 51.31 ± 0.87 | 52.67 ± 0.58 | 49.20 ± 0.45 |
| Example 2 | 54.23 ± 0.37 | 55.16 ± 0.27 | 55.92 ± 0.96 | 57.21 ± 0.60 | 53.83 ± 0.29 |
| Example 4 | 43.76 ± 0.65 | 43.24 ± 0.36 | 43.44 ± 0.26 | 43.57 ± 0.57 | 12.35 ± 0.10 |
| Example 5 | 46.36 ± 0.31 | 47.91 ± 0.71 | 47.42 ± 2.38 | 46.17 ± 0.31 | 41.81 ± 0.07 |
| Example 6 | 25.87 ± 0.24 | 29.34 ± 0.64 | 29.66 ± 0.91 | 28.16 ± 2.23 | 9.19 ± 0.26 |

1. A rapidly soluble tacrolimus solid dispersion formulation comprising tacrolimus and enteric polymer, wherein the weight ratio of tacrolimus and the enteric polymer is in the range of 1:0.8˜1.2, and the latter is selected from the group consisting of hydroxypropylmethylcellulose phthalate, enteric acrylate copolymer, hydroxypropylmethylcellulose acetate succinate or carboxymethylethylcellulose.
